SEQUENCE LISTING (1 sequence)

SEQ ID NO: 1 - DNA, 15 bp, Artificial Sequence
  Description of Artificial SequenceSynthetic DNA
ggttggtgtggttgg

(19) United States
(12) Patent Application Publication
(10) Pub. No.: US 2006/0223170 A1
(43) Pub. Date: Oct. 5, 2006

(54) Biomolecule detecting apparatus and biomolecule detecting method employing the same

(76) Inventors: Masao Kamahori, Kokubunji (JP); Yu Ishige, Tokyo (JP); Maki Shimoda, Hino (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/329,086

(22) Filed: Jan. 11, 2006

(30) Foreign Application Priority Data
Mar. 29, 2005 (JP) 2005-095675

(51) Int. Cl. C12M 1/34

(52) U.S. Cl. 435/287.2

(57) ABSTRACT

A biomolecule detecting element capable of easily immobilizing a detecting probe and being used in a simple manner without requiring a dark box and the like. A biomolecule detecting probe is immobilized on the surface of a conductive electrode of an insulated gate field effect transistor, the conductive electrode being formed on the surface of a gate insulating material between a source and a drain. Portions other than the conductive electrode are covered with a light-shielding member so as to eliminate the influence of light. During measurement, the conductive electrode having a biomolecule detecting probe immobilized on the surface thereof and a reference electrode are disposed in a buffer solution in a measurement cell. A rectangular wave is applied to the reference electrode from a power supply, and a response waveform that changes before and after the binding of a measurement target, such as DNA or protein contained in the buffer solution, to the biomolecule detecting probe, namely, a change in the value of current that flows between the source and the drain, is detected.

### CLAIM OF PRIORITY

The present application claims priority from Japanese application JP 2005-095675 filed on Mar. 29, 2005, the content of which is hereby incorporated by reference into this application.

### BACKGROUND OF THE INVENTION

1. Field of the Invention

The present invention relates to a detection apparatus and method for measuring bio-related substances, such as DNA and proteins in particular, without labeling. Particularly, it relates to a detection apparatus and method employing a field-effect transistor.

2. Background Art

The significant progress in base sequencing techniques that has been made in recent years has resulted in the sequencing of substantially all of the base sequences of the human genome. There is now a growing movement to utilize the resultant DNA base sequence information widely, such as in medicine, for example. It is expected that in the future, light will be shed on diseases on an individual level or individual predispositions on a genetic level, so that great progress will be made in “made-to-order medicine” tailored for each individual predisposition. Other significant progress is also expected in a wide-ranging field other than medicine or medication, such as breed improvement in agricultural products. At the base of these progress lies gene expression information and functional information as well as base sequence information. Gene function and expression analyses are currently being carried out on a large scale using DNA chips, and databases are being created. However, because the current DNA chips employ fluorescence detection method as their basic principle, they require laser light sources and complex optical systems, resulting in a large and expensive measurement system. Although suitable for processing a large quantity of samples, such a system is not suitable for measuring a small number of samples in a small-scale measuring site. Thus, there is a need for a small, easy-to-operate measurement apparatus suitable for small-scale measurement sites where a growing demand is expected.

In response to such need, there have been reported a DNA chip of amperometric detection type that employ oxidation/reduction marker substance, or a DNA sensor of potentiometric detection type that utilizes electric characteristics of transistor. These DNA chips for electrical measurement do not require laser light sources or complex optical systems and facilitate the reduction in size of apparatus.

In the amperometric detection type that employs oxidation/reduction marker substance, a property is utilized that the oxidation/reduction substance is intercalated between the double-stranded DNA that is formed when a target DNA is bound (hybridized) to a DNA probe. The presence or absence of the binding (hybridization) between the target DNA and the DNA probe is determined by detecting the exchange of electrons between the intercalated oxidation/reduction substance and a metal electrode as a current change (namely, an oxidation/reduction current) (Analytical Chemistry 66, (1994) 3830-3833).

On the other hand, in the potentiometric detection type utilizing electrical characteristics of the transistor, a DNA probe is immobilized on a gate insulating layer formed on top of a source electrode and a drain electrode, and the surface potential (namely, surface charge density) on an insulating film produced by the binding (hybridization) of a target DNA to the DNA probe is detected as a change in current value between the source electrode and the drain electrode (JP Patent Publication (Kohyo) No. 2001-511245 A). The gate insulating material consists of silicon oxide, silicon nitride, or tantalum oxide, for example, either individually or in combination. Normally, in order to maintain a good transistor operation, silicon nitride, tantalum oxide and the like are layered on silicon oxide and the like, thereby forming a dual structure. For the immobilization of a DNA probe on the gate insulating layer, the surface of the gate insulating layer is chemically modified with aminopropylsilane or polylysine, for example, so as to introduce an amino group, and then the DNA probe with its terminal chemically modified with the amino group is reacted using glutalaldehyde or phenylenediisocyanate.

Non-patent Document 1: Analytical Chemistry 66, (1994) 3830-3833

Patent Document 1: JP Patent Publication (Kohyo) No. 2001-511245 A

### SUMMARY OF THE INVENTION

In the amperometric detection type using an oxidation/reduction marker substance, because it is based on the basic principle of detecting oxidation/reduction current on the metal electrode, a current due to a coexisting substance flows if there is a coexisting oxidizing or reducing substance in the sample, which interferes with gene detection. Furthermore, the current measurement is associated with the progress of electrochemical reaction on the surface of the metal electrode, resulting in the corrosion of the electrode or the gas generation. This will destabilize measurement conditions and lead to deterioration of detection sensitivity and accuracy. It is also difficult to use this system for the measurement of biological molecules other than DNA because it generally employs an intercalator as the oxidation/reduction marker substance.

On the other hand, in the potentiometric detection type utilizing electrical characteristics of the transistor, the corrosion of the insulating layer on the chip, gas generation, or the interference from coexisting oxidation/reduction substance and the like does not pose much of a problem as compared with the amperometrict detection type. However, in the structure adopted in this type, the insulating layer doubles as a sensing portion, resulting in the need to carry out complex preprocessing, such as silane coupling, for the immobilization of the DNA probe to the gate insulating layer. Further, because the layer including the gate insulating layer for immobilizing the DNA probe causes measurement error in response to light, the system requires a light-shielding box for measurement. In addition, because this measurement system measures a potential change on the gate insulating layer as a drain current change, the system must wait before the potential on the gate insulating layer, namely the sensing portion, stabilizes.

It is therefore an object of the invention to provide a biomolecule detecting apparatus that is capable of easily immobilizing a detecting probe and simple to use, requiring no light-shielding box and the like.

In order to achieve the aforementioned object, the invention provides a biomolecule detecting apparatus in which an electrically conductive electrode for immobilizing a detection probe is connected to the gate of an insulated gate field effect transistor by an electrically conductive wire. By adopting such a structure, it becomes possible to shield the gate portion of the insulated gate field effect transistor from light without covering the electrically conductive electrode for immobilizing the probe, which is a sensing portion, with light-shielding material. In addition, by using gold in the electrically conductive electrode, the detection probe, which is provided wit alkanethiol at the terminal thereof, can be immobilized by a simple operation, namely, by adding or spotting a detection probe solution dropwise onto the surface of the gold electrode.

With regard to the instability of surface potential due to external fluctuations (or drift), which poses a problem when the electrically conductive electrode is used within a solution, such influence can be reduced by measuring the response when an input waveform such as a rectangular wave is applied across the conductive electrode and a reference electrode. Such an application of voltage of, e.g., rectangular waveform does not break the binding between the detection probe and the measurement target. Use of precious metal, such as gold, does not cause a reaction on the surface of the electrode within the solution.

In accordance with the invention, an insulated gate field effect transistor having a conductive electrode, on the surface of which a detection probe is immobilized as a biomolecule detecting element, is used to measure a change in the electrical characteristics of the transistor before and after the binding between the measurement target and the biomolecule detecting probe, in terms of a response to an input waveform such as a rectangular wave. In this way, the presence or absence of a measurement target, such as DNA or protein that may be contained in a sample solution can be detected while reducing the influence of external fluctuations. The influence of light that poses a problem during detection can be easily eliminated by shielding the transistor except for its electrode, which constitutes a sensing portion.

### BRIEF DESCRIPTION OF THE DRAWINGS

FIG. 1 shows an example of an insulated gate field effect transistor used in a biomolecule detecting apparatus of the invention, with (a) showing a schematic cross-section and (b) showing a schematic plan view thereof.

FIG. 2 shows a block diagram of a biomolecule detecting apparatus employing a biomolecule detecting element of the invention.

FIG. 3 shows a light-shielding effect of the insulated gate field effect transistor used in the biomolecule detecting apparatus of the invention, with (a) showing the result of measurement obtained using an insulated gate field effect transistor with no light-shielding measure taken while (b) showing the result of measurement obtained using the element of the invention.

FIG. 4 shows a method for controlling the sequence of DNA and immobilizing it on the surface of a gold electrode, with (a) showing a state where a single-stranded DNA is immobilized while (b) showing a state where a double-stranded DNA is formed on the surface of the gold electrode.

FIG. 5 shows a waveform analysis method using a biomolecule detecting element of the invention, with (a) showing the waveform of an applied voltage while (b) showing the waveform of a drain current.

FIG. 6 shows an example in which a single-stranded DNA and a double-stranded DNA are detected using a biomolecule detecting method of the invention.

FIG. 7 shows an example of a waveform analysis method using a biomolecule detecting element in another embodiment of the invention, where the input waveform is sinusoidal.

FIG. 8 shows the result of detecting the presence or absence of complementary DNA in a solution based on the difference in response between a single-stranded DNA and a double-stranded DNA, using a biomolecule detecting apparatus in another embodiment of the invention where a sine wave is used as an input waveform.

FIG. 9 shows an example of the structure of an insulated gate field effect transistor according to another embodiment of the invention, where a sample measuring electrode and a control electrode are combined on the same element.

FIG. 10 shows a schematic cross-section of a biomolecule detecting element of differential type according to another embodiment of the invention, in which a reference element is combined.

FIG. 11 shows a measurement method adapted for the biomolecule detecting element of differential type according to another embodiment of the invention in which a reference electrode is combined.

### DESCRIPTION OF PREFERRED EMBODIMENTS

Embodiments of the invention will be described with reference to the drawings.

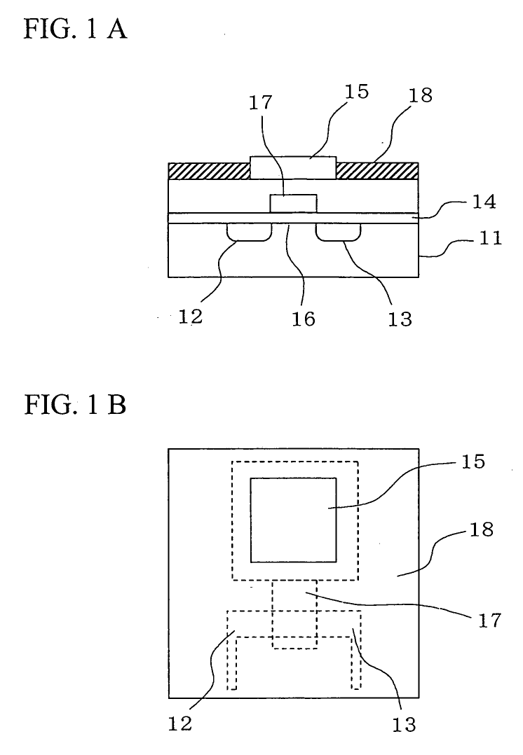

FIG. 1 shows an example of the structure of an insulated gate field effect transistor used in a biomolecule detecting apparatus of the invention. FIG. 1(a) shows a schematic cross-section, and FIG. 1(b) shows a schematic plan view. The insulated gate field effect transistor includes a source 12, a drain 13, and a gate insulating material 14 formed on the surface of a silicon substrate 11, and a conductive electrode 15. The conductive electrode, on which a detection probe is immobilized, is connected with the gate 16 of the insulated gate field effect transistor by a conductive wire 17. Portions other than the conductive electrode 15 are covered with a light-shielding member 18, which can be made of plastic material or glue with low optical transparency. Alternatively, an aluminum layer may be formed during the semiconductor manufacturing process. By adopting this structure, the apparatus can be used in a simple manner without requiring a light-shielding box and the like. Preferably, the insulated gate field effect transistor is comprised of a metal-insulator-semiconductor field effect transistor (FET) in which a silicon oxide is used as an insulating film. Alternatively, a thin-film transistor (TFT) may be used without any problems.

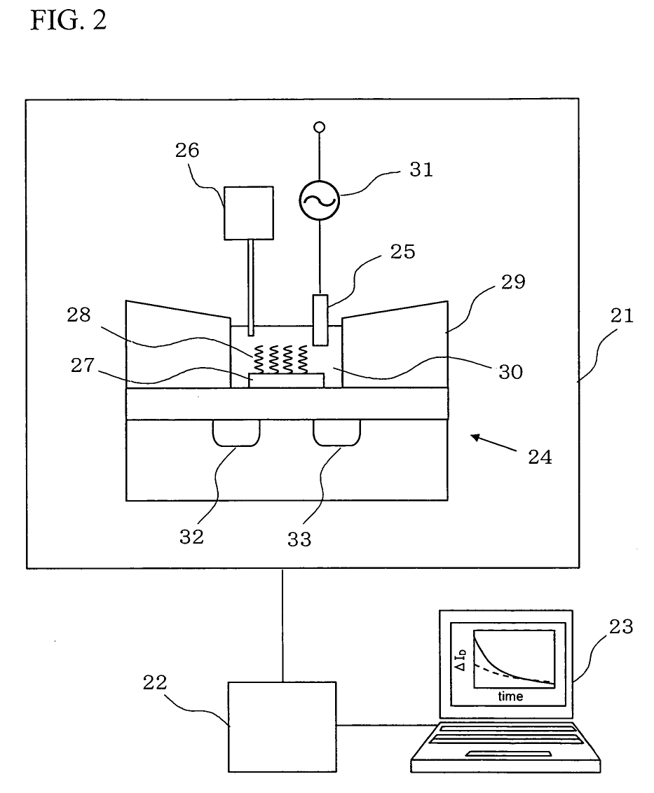

FIG. 2 shows a block diagram of the biomolecule detecting apparatus using the biomolecule detecting element according to the invention. The measurement system of the invention is comprised of a measurement unit 21, a signal processing circuit 22, and a data processing device 23. In the measurement unit 21, there are disposed an insulated gate field effect transistor 24, a reference electrode 25, and a sample injector 26.

A measurement procedure is as follows. Initially, the conductive electrode 27, a biomolecule detecting probe 28 immobilized on the surface of the conductive electrode 27, and the reference electrode 25 are placed in a buffer solution 30 in a measurement cell 29. A power supply 31 is used to apply a rectangular or sine wave voltage to the reference electrode 25, and the response is measured as a current change between the source 32 and the drain 33. The response characteristics are then recorded using the signal processing circuit 22 and the data processing device 23. Then, a sample is introduced into the buffer solution 30 in the measurement cell 29, using the sample injector 26. Thereafter, a rectangular or sine wave voltage from the power supply 31 is applied to the reference electrode 25, and the response is measured as a change in the current between the source 32 and the drain 33. The response characteristics are recorded using the signal processing circuit 22 and the data processing device 23. As the buffer solution, a Tris-HCL buffer (10 mM Tris-HCl, 5 mM Mg, pH 7.2) is used.

When a biological substance in the introduced sample binds to the biomolecule detecting probe 28, the surface condition of the conductive electrode 27 changes, whereby the response characteristics of the insulated gate field effect transistor 24 change with respect to the rectangular wave or sine wave applied to the reference electrode 25. Therefore, by determining whether or not the response characteristics of the insulated gate field effect transistor 24 have changed in response to the application of the rectangular wave or sine wave voltage before and after the introduction of sample, it can be determined whether or not the biological substance has bound to the biomolecule detecting probe 28, namely, whether or not the target DNA or protein is included in the sample.

The biomolecule detecting probe 28 may employ a nucleic acid such as a single-stranded DNA fragment, protein or peptide such as antibody, antigen, or enzyme, or sugars, for example. The selectivity of the biomolecule detecting probe is based on the difference in specific affinity due to the inherent structure of a biological component. When the detection target is DNA, the biomolecule detecting probe employs a single-stranded DNA fragment having a sequence complementary to the detection target DNA. In this case, the length of the single-stranded DNA fragment is normally 20 to 50 bases long. While an antibody or antigen and the like can be used as is, it is also possible to use a single-stranded DNA fragment called “aptamer” instead of them. For example, the aptamer for α-thrombin, which is a type of blood-coagulating serine protease, is 5′-GGTTGGTGTGGTTGG-3′.

The reference electrode 25 provides a reference potential for the stable measurement of potential change that occurs on the surface of the conductive electrode 27 in the sample solution 30 due to equilibrium reaction or chemical reaction. Normally, the reference electrode is comprised of a silver/silver chloride electrode or a calomel electrode having saturated potassium chloride as an internal solution. When the composition of the measured sample solution is constant, however, a silver/silver chloride electrode alone can be used as a pseudo-electrode without any problems.

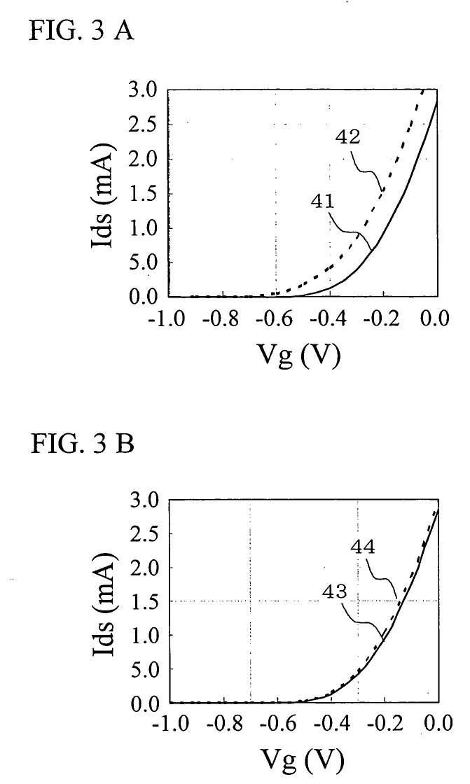

FIGS. 3(a) and (b) shows a light-shielding effect of the insulated gate field effect transistor used in the biomolecule detecting apparatus of the invention. As a light-shielding member, an aluminum layer was formed on a silicon oxide layer prior to the final step of the semiconductor manufacturing process (namely, the formation of a silicon nitride layer). For the evaluation of the light-shielding effect, the measurement results of current and voltage characteristics of the insulated gate field effect transistor were compared in terms of the presence or absence of a light-shielding box. For the measurement of the current and voltage characteristics of the transistor, a semiconductor parameter analyzer (Agilent 4155C Semiconductor Parameter Analyzer) was used, with the source-drain voltage set at 0.5 V and using an Ag/AgCl reference electrode as the reference electrode.

FIG. 3(a) shows the result of measurement using the insulated gate field effect transistor when no light-shielding measure was taken. FIG. 3(b) shows the result of measurement using the insulated gate field effect transistor according to the invention wherein a light-shielding measure was taken by covering the portions of the transistor other than the electrode portion, which is the sensing portion, with a light-shielding member. The results show that, in the case of the insulated gate field effect transistor without any light-shielding measure taken, there was a large difference between the drain current value 41 in the case where the transistor was placed inside the light-shielding box and the drain current value 42 in the case where no light-shielding box was used, as shown in FIG. 3(a). In the case of the insulated gate field effect transistor of the invention where the light-shielding measure was taken, there was little difference between the drain current value 43 when the transistor was placed inside the light-shielding box and the drain current value 44 when no light-shielding box was used, as shown in FIG. 3(b), thus indicating the absence of influence from light.

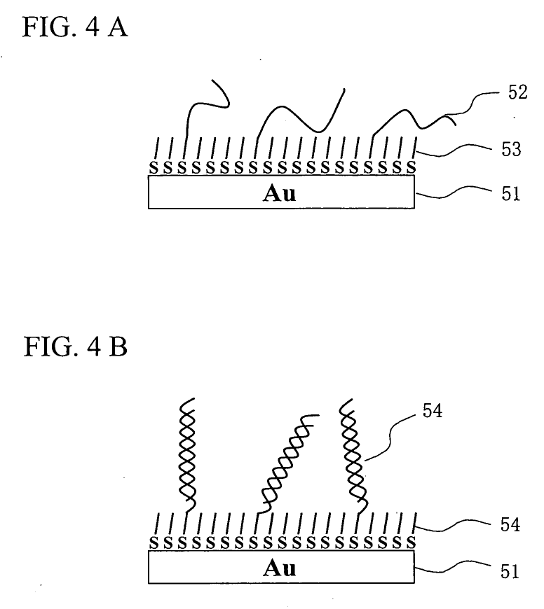

In the present example, the conductive electrode was comprised of a gold thin film 51 and the biomolecule detecting probe was comprised of a single-stranded DNA 52. As shown in FIG. 4(a), when the DNA probe 52 was immobilized to the surface 51 of the gold thin film, alkanethiol 53 was simultaneously immobilized so as to control the orientation of the DNA probe 52 and to protect the surface of the gold thin film 51. When DNA is immobilized, because DNA is negatively charged, the DNA fragment is caused to lie flat on the surface by interaction if alkanethiol used includes an amino group. This would reduce measurement stability (fluctuations in stabilization time and measured values). Therefore, alkanethiol should be used that includes a hydroxyl group or a carboxyl group. Examples of alkanethiol that can be used include mercaptoethanol, 6-hydroxy-1-hexanethiol, 8-hydroxy-1-octanethiol, and 11-hydroxy-1-undecanethiol, having a hydroxyl group as the terminal group. The terminal group may be either an amino group, a carboxyl group, or a hydroxyl group depending on the charge possessed by the measurement target. If the physical adsorption onto the electrode surface presents a problem, a fluorocarbon group and the like may be used. After the sensor portion is disposed in the sample solution, the DNA probe 52 and a single-stranded DNA with a complementary sequence are injected into the sample solution, whereby a double-stranded DNA 54 is formed, as shown in FIG. 4(b).

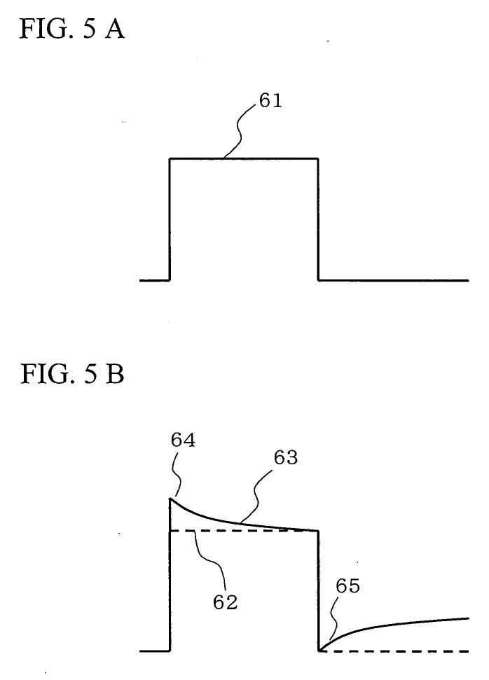

In the following, the principle of measurement of the invention is described. FIG. 5 shows a waveform analysis method using the biomolecule detecting element of the invention. FIG. 5(a) shows the waveform of the voltage applied to the reference electrode. FIG. 5(b) shows the waveform of a drain current. The insulated gate field effect transistor exhibits a response indicated by a broken line 62 in FIG. 5(b) with respect to an input waveform 61 shown in FIG. 5(a). When the biomolecule detecting probe is immobilized on the conductive electrode, the response of the biomolecule detecting probe is added to this response, creating a response waveform indicated by a solid line 63 in FIG. 5(b). Thus, the amount of change in a relaxation component 64 in the rise portion of the response waveform or that in a relaxation component 65 in the fall portion of the response waveform is measured so as to detect the change in the state of the biomolecule detecting probe. By measuring the change in response to the applied voltage waveform, the influence of external fluctuations can be reduced.

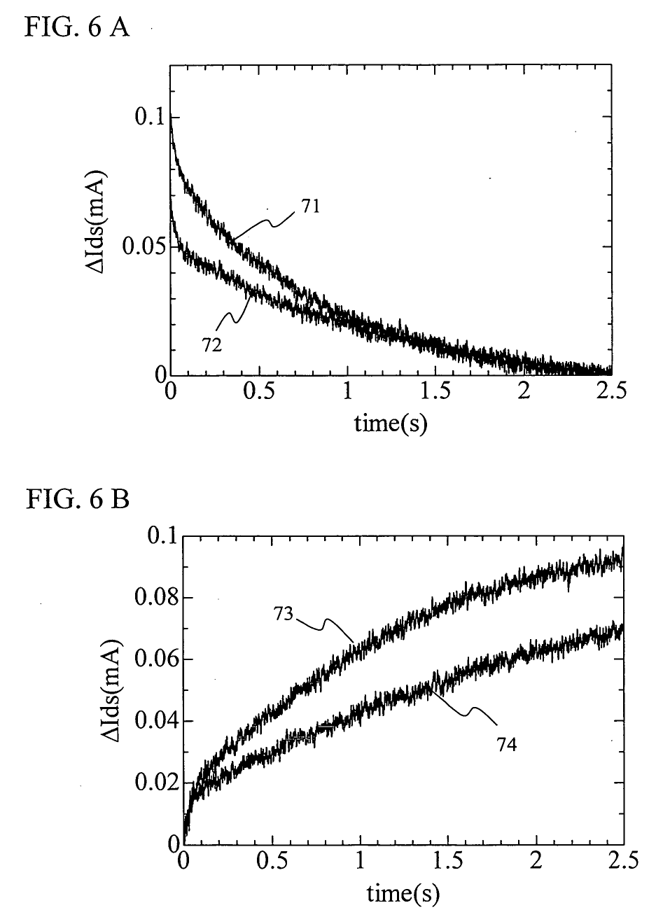

FIG. 6 shows an example in which the presence or absence of a complementary DNA in the solution was detected on the basis of the difference in response between a single-stranded DNA and a double-stranded DNA, using the biomolecule detecting apparatus of the invention. The DNA probe was comprised of DNA having 30 bases (AAAAA AAA .. ..... ...... .. AAA AAAAA). The detection target had a sequence complementary to the DNA probe (TTTTT TTT .. ..... ..... .. TTT TTTTT)). The reference electrode was comprised of an Ag/AgCl reference electrode, and a rectangular voltage of 0.2 Hz with Vₘₐₓ=0 V and Vₘᵢₙ=−0.3 V was applied, using a function generator. The source-drain voltage was 1 V, and the drain current was converted into voltage by a signal processing circuit, and the resultant waveform was loaded onto a PC using a digital-analog converter (DAC).

FIG. 6(a) shows a response component of the output waveform at the rise of input voltage, while FIG. 6(b) shows that of the output waveform at the fall of input voltage. As compared with the response waveforms 71 and 73 prior to the introduction of the complementary-strand sequence into the sample solution, it can be seen that the response waveforms 72 and 74 after the introduction of the complementary-strand sequence are smaller. This change in response reflects the formation of a double strand by the DNA probe due to the introduction of the complementary-strand sequence. Because the double-stranded DNA has formed a double strand, it has greater rigidity than single-stranded DNA and it therefore has a smaller response to the change in applied voltage. As a result, the magnitude of output signal of double-stranded DNA is smaller than that of single-stranded DNA. With regard to the frequency response of DNA, it responds up to approximately 1 kHz, so that a rectangular wave having a repetition frequency of 1 kHz or below can be used as an input waveform without any problems. Preferably, the repetition frequency is 10 Hz or below, for this makes response analysis easier.

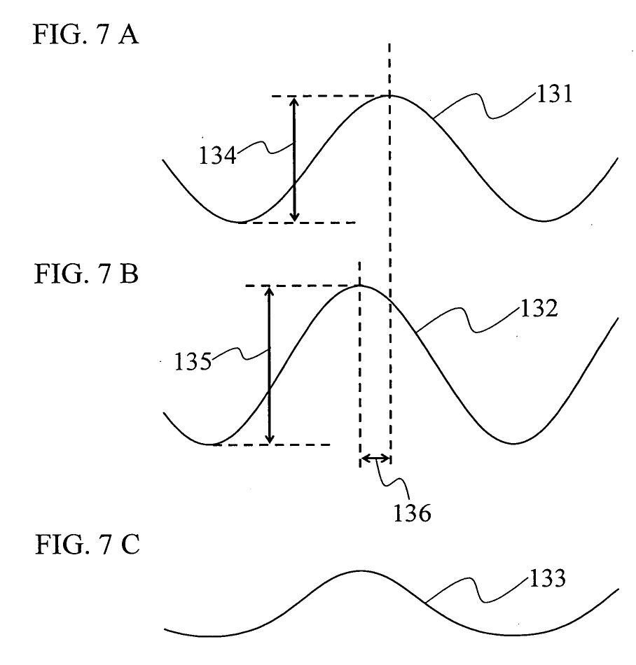

FIG. 7 shows an example of the waveform analysis method using the biomolecule detecting element of the invention where a sine wave was used as the input waveform. FIG. 7(a) shows the waveform of the applied voltage to the reference electrode, and FIG. 7(c) shows the waveform of the drain current. The response of the drain current to the gate voltage of the FET is not linear, so that, when a sine wave is inputted, the drain current exhibits a distorted sine waveform. Therefore, when the output waveform is converted into an input voltage in accordance with the voltage/current characteristics of a separately measured FET, a sine wave without distortion can be obtained as shown in FIG. 7(b). Amplitude 135 of the output waveform is smaller than amplitude 134 of the input waveform, and there is also a phase shift 136. Such changes in amplitude and phase have to do with the measurement using a rectangular wave and are related by the following equation.

When a change in the input waveform affects the output waveform, the output waveform g(t) can be expressed by the following equation, using the input waveform f(t) and a response function h(t):

$$g ( t ) = f ( t ) + {\int }_{0}^{∞} \frac{ⅆ f ( t - {t}^{′} )}{ⅆ t} h ( {t}^{′} ) ⅆ {t}^{′} \qquad ( 1 )$$

When the input waveform f(t) is a step-like function corresponding to the rise of the rectangular wave, namely, the following equation (2), we have equation (3). Therefore, equation (1) becomes equation (4), where h(t) means a relaxation component and can be experimentally determined.

$$\begin{matrix}f ( t ) = \{ \begin{matrix}0 & ( t < 0 ) \\ 1 & ( t \geq 0 )\end{matrix} & ( 2 ) \\ \frac{ⅆ f ( t )}{ⅆ t} = δ ( t ) & ( 3 ) \\ \begin{matrix}g ( t ) = f ( t ) + {\int }_{0}^{∞} δ ( t - {t}^{′} ) h ( {t}^{′} ) ⅆ {t}^{′} \\ = f ( t ) + h ( t )\end{matrix} & ( 4 )\end{matrix}$$

Response to a sine wave can be determined by f(t)=sin(ωt). Thus, the same measurement can be made using a sine wave as when a rectangular wave is used. Namely, by measuring the change in amplitude and phase when a sine wave is inputted, the change in the state of the biomolecule detecting probe can be measured.

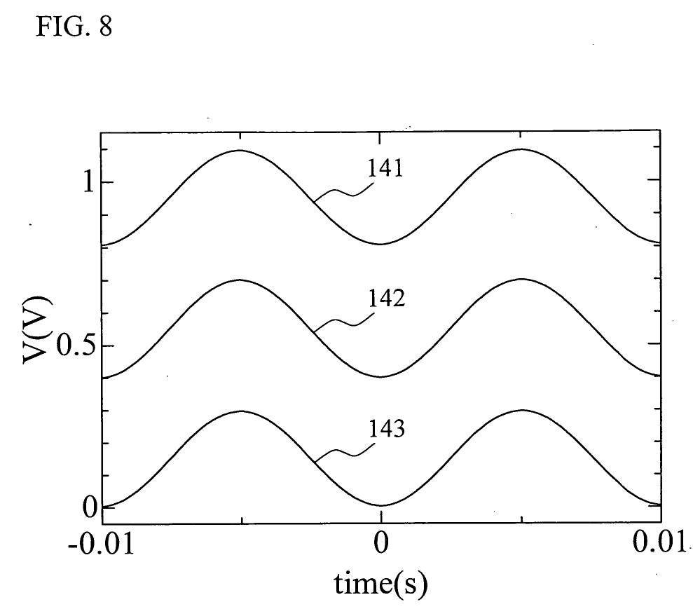

FIG. 8 shows an example in which, using the biomolecule detecting apparatus of the invention with a sine wave as input, the presence or absence of a complementary-chain DNA in a solution was detected based on the difference in response between a single-stranded DNA and a double-stranded DNA. The DNA probe was comprised of DNA of 30 bases (AAAAA AAA.. ..... ..... ..AAA AAAAA), and the detection target was comprised of a complementary sequence to the DNA probe (TTTTTT TTT .. ..... ..... .. TTT TTTTT). The reference electrode was comprised of an Ag/AgCl reference electrode, and a sine wave voltage of 100 Hz, Vₘₐₓ=0V, and Vₘᵢₙ=−0.3V was applied using a function generator. The source-drain voltage was 1V, and the drain current was converted into a voltage by a signal processing circuit, and the resultant waveform was loaded into a PC using a DAC (digital-analog converter).

Against an input waveform 141, output waveforms 142 and 143 were obtained by converting the drain current into a voltage in accordance with the voltage/current characteristics of the FET. The output waveform 142 is the waveform prior to the introduction of the double-stranded DNA, while the output waveform 143 is the waveform after the introduction of the double-stranded DNA. These waveforms have a substantially identical phase. On the other hand, with regard to amplitude, the output waveform 142 was 1.010 and the output waveform 143 was 1.006 against the input waveform 141 of 1. The change in response reflects the formation of a double strand by the DNA probe as a result of the introduction of the complementary-strand sequence. Because the double-stranded DNA formed a double strand, it has greater rigidity than the single-stranded DNA and a smaller response to the change in applied voltage. Accordingly, the magnitude of the output signal of the double-stranded DNA is smaller than that of the single-stranded DNA.

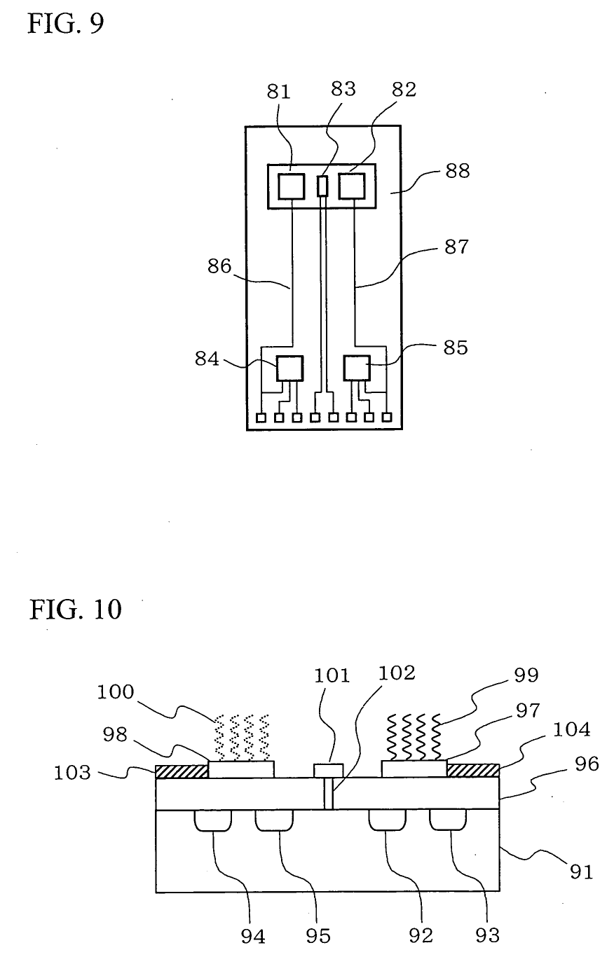

FIG. 9 shows another embodiment of the invention in which a sample measuring electrode and a control electrode are mounted together on a single element. Normally, the presence or absence of a measurement target can be detected by comparing the measured values before and after the binding of the measurement target to the detecting probe. In some case, however, impurities present in the measured solution may bind to the detecting probe or become physically adsorbed on the surface of the gold electrode. In such cases, the measured values before and after the binding of the measurement target to the detecting probe are affected, leading to a drop in measurement accuracy. In accordance with the present embodiment, a differential measurement is carried out between a measurement transistor and a reference transistor. As a result, the influence of output fluctuations or ambient temperature caused by the non-specific adsorption of impurities other than the measurement target can be cancelled or corrected for, so that the measurement target alone can be measured accurately.

The element in the present embodiment is comprised of a sample measuring electrode 81, a control electrode 82, and a temperature measuring diode 83 that are mounted together. In this element, which is an extended gate and depletion-type FET using an SiO₂ insulating layer (thickness of 17.5 nm), the sample measuring electrode 81 and the control electrode 82 are connected to gates 84 and 85, respectively, of an insulated gate field effect transistor by conductive wires 86 and 87. The electrodes 81 and 82 are each comprised of a gold electrode measuring 400 μm×400 μm formed on an extended and enlarged gate. At portions other than the electrodes 81 and 82, an aluminum layer was formed below silicon nitride as a light-shielding member 88. Because measurement normally involves an aqueous solution, the element of the present embodiment must be operable in a solution. When measurement is made in a solution, the element needs to operate within an electrode potential range where electrochemical reaction is hard to occur, namely, between −0.5 and 0.5V. For this reason, in the present embodiment, the manufacturing conditions for the depletion type n-channel FET, namely, the ion implantation conditions for the adjustment of threshold voltage (Vₜ), are adjusted so as to set the threshold voltage of the FET at near −0.5V. The temperature measuring diode mounted in the present element was of the n⁺/p junction type. The temperature characteristics of the n⁺/p junction diode manufactured in the present example were such that the temperature coefficient was approximately 1.8 mV/° C.

One advantage of the extended-gate FET of the embodiment is that the sensing portion can be designed to have any desired dimensions and located at any desired site depending on the measurement target. Furthermore, because a probe for a particular measurement target can be immobilized in the final step using chips manufactured in the same process, common steps can be adopted for manufacturing sensors for a variety of measurement targets. The gold electrode for immobilizing the probe in accordance with the present embodiment can easily bind to a thiol compound and is stable. Therefore, by using a probe having a thiol group (normally, alkanethiol linker), immobilization can be facilitated. In addition, the gold electrode is inactive and therefore stable in a solution, i.e., it does not produce potential drift and the like.

A biomolecule detecting element of the differential type according to another embodiment of the invention is described with reference to FIG. 10, in which a reference element is mounted together. FIG. 10 shows a schematic cross-section showing the components of the embodiment, such as transistors, conductive electrodes, and light-shielding member, arranged in a manner similar to the embodiment shown in FIG. 9.

In the element of the present embodiment, a source 92 and drain 93 of a measurement transistor and a source 94 and drain 95 of a reference transistor, as well as gate insulating material 96 are formed on the surface of a silicon substrate 91. Conductive electrodes 97 and 98 are formed on the surface of the gate insulating material between the source 92 and drain 93 of the measurement transistor, and between the source 94 and drain 95 of the reference transistor, respectively. On the surface of the conductive electrodes 97 and 98, there are immobilized a biomolecule detecting probe 99 and a pseudo-molecule detecting probe 100. For example, in the case of DNA measurement, the biomolecule detecting probe 99 is comprised of a DNA probe having a base sequence complementary to a target gene, while the pseudo-molecule detecting probe 100 is comprised of a DNA probe having a base sequence different from the sequence complementary to the target gene. In the same plane as that of the conductive electrodes 97 and 98, there is provided a pseudo-reference electrode 101 which is connected to the outside via a conductive wire 102. The pseudo-reference electrode may be made of silver/silver chloride, gold, or platinum, for example. At portions other than the electrodes 97 and 98, an aluminum layer is formed below silicon nitride as light-shielding member 103 and 104.

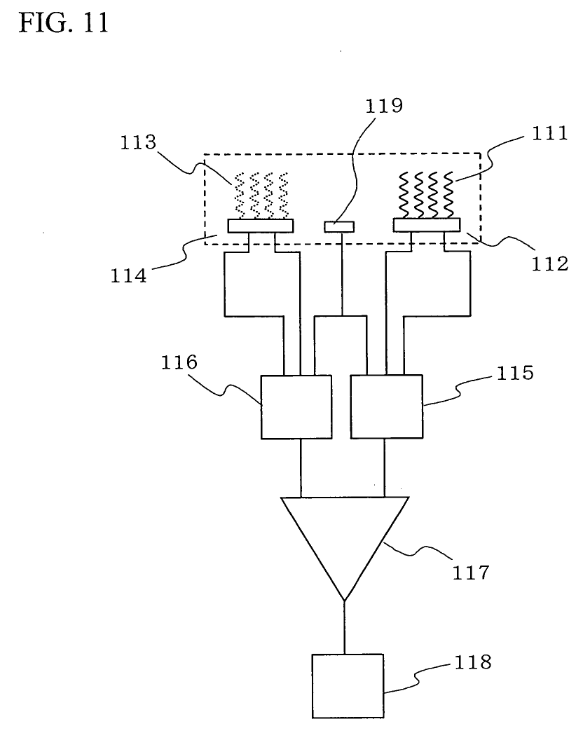

In actual measurement, as shown in FIG. 11, the output of a measurement transistor 112 having a DNA probe 111 immobilized thereon that has a base sequence complementary to the target gene, and the output of a reference transistor 114 having a DNA probe 113 immobilized thereon that has a base sequence different from the base sequence complementary to the target gene, are inputted to transistor drive circuits 115 and 116, respectively. The surface potential of each is then measured, and the outputs are inputted to a signal processing circuit 118 via a differential amplification circuit 117. In order to measure the measurement transistor 112 and the reference transistor 114 stably, a common reference electrode 119 is provided that serves as a reference for potential measurement. In the present example, measurement was taken by applying a DC voltage of 1.0V between the source and drain and applying a rectangular wave voltage of Vₘₐ=0V and Vₘᵢₙ=−0.3V with a repetition frequency of 0.2 Hz to the reference electrode (Ag/AgCl reference electrode) on the gate side.

While the reference electrode was made of silver/silver chloride, gold or platinum and the like can also be used without any problems. By thus carrying out a differential measurement using a measurement transistor and a reference transistor, output value fluctuations due to the influence of ambient temperature, or output fluctuations due to the non-specific adsorption of impurities other than the measurement target onto the surface of the conductive electrode, can be cancelled or corrected. As a result, the measurement target alone can be accurately measured. In addition, by combining the differential measurement and the pseudo-reference electrode, changes in solution composition can be corrected, so that a small-sized and wholly solid detecting element can be realized.

What is claimed is:

1. A biomolecule detecting apparatus comprising:
a field effect transistor;
an electrode connected to the gate of said field effect transistor by a wire, wherein said electrode is in contact with a buffer solution into which a sample is introduced and has a probe that binds to a target in said sample immobilized on the surface thereof;
a reference electrode that comes into contact with said buffer solution;
a power supply for applying an input voltage waveform having a frequency of 1 kHz or lower across said electrode and said reference electrode; and
a detecting unit for detecting a change in response of said field effect transistor with respect to said input voltage waveform.

2. The biomolecule detecting apparatus according to claim 1, wherein the frequency of said input voltage waveform is 10 Hz or lower.

3. The biomolecule detecting apparatus according to claim 1, wherein said input voltage waveform is rectangular, and wherein said detecting unit detects a change in the rise or fall portion of a response waveform of said field effect transistor.

4. The biomolecule detecting apparatus according to claim 1, wherein said input voltage waveform is sinusoidal, and wherein said detecting unit detects a change in a response waveform of said field effect transistor.

5. The detecting apparatus according to claim 1, wherein said electrode is made of gold.

6. The biomolecule detecting apparatus according to claim 1, further comprising a second field effect transistor, and a second electrode that is connected with the gate of said second field effect transistor by a wire, wherein said second electrode is in contact with said buffer solution and has a probe that does not bind to a target in said sample immobilized on the surface thereof,
wherein said detecting unit comprises a differential amplifier to which an output of said field effect transistor and an output of said second field effect transistor are inputted.

7. The biomolecule detecting apparatus according to claim 1, wherein the source, drain, and channel of said field effect transistor are covered with a light-shielding member.

8. The biomolecule detecting apparatus according to claim 1, wherein said probe comprises a nucleic acid, antibody, antigen, or enzyme.

9. The biomolecule detecting apparatus according to claim 8, wherein said probe is immobilized on the surface of said electrode via alkanethiol coupled to one end of said probe.

10. A biomolecule detecting apparatus comprising:
a field effect transistor:
an electrode connected to the gate of said field effect transistor by a wire, wherein said electrode is in contact with a buffer solution into which a sample is introduced and has a probe that binds to a target in said sample immobilized on the surface thereof;
a reference electrode that comes into contact with said buffer solution;
a power supply connected to said reference electrode; and
a detecting unit for processing an output of said field effect transistor,
wherein the source, drain, and channel of said field effect transistor are covered with a light-shielding member.

11. The biomolecule detecting apparatus according to claim 10, wherein said light-shielding member comprises an electrically conductive member.

12. The biomolecule detecting apparatus according to claim 11, wherein said electrically conductive member is grounded.

13. The biomolecule detecting apparatus according to claim 11, wherein said electrically conductive member is aluminum or gold.

14. A method for detecting a biomolecule comprising the steps of:
bringing a buffer solution into contact with an electrode of a field effect transistor, said electrode having a probe that binds to a target in a sample immobilized on the surface thereof;
applying an input voltage waveform across said electrode and a reference electrode that is in contact with said buffer solution;
injecting a sample into said buffer solution; and
detecting a change in response of said field effect transistor before and after the injection of said sample.

15. The method for detecting a biomolecule according to claim 14, wherein said electrode is connected to the gate of said field effect transistor by a wire.

16. The method for detecting a biomolecule according to claim 14, wherein the frequency of said input voltage waveform is 1 kHz or lower.

17. The method for detecting a biomolecule according to claim 14, wherein said probe comprises a nucleic acid, antibody, antigen, or enzyme.

18. The method for detecting a biomolecule according to claim 14, wherein said input voltage waveform is rectangular, and wherein a change in the rise or fall portion of a response waveform of said field effect transistor before and after the injection of said sample is detected.

19. The method for detecting a biomolecule according to claim 14, wherein said input voltage waveform is sinusoidal, and wherein a change in a response waveform of said field effect transistor before and after the injection of said sample is detected.
